(19) United States
(12) Patent Application Publication
(10) Pub. No.: US 2008/0075679 A1
(43) Pub. Date: Mar. 27, 2008

(54) Compositions containing dark glitter

(75) Inventors: Sue Feng, Edison, NJ (US); Diane Keene, Burlington, NJ (US); Ana Kljuic, New York, NY (US)

Correspondence Address: OBLON, SPIVAK, MCCLELLAND MAIER & NEUSTADT, P.C., 1940 DUKE STREET, ALEXANDRIA, VA, 22314, US

(73) Assignee: L'Oréal, Paris (FR)

(21) Appl. No.: 11/527,416

(22) Filed: Sep. 27, 2006

(51) Int. Cl. A61K 8/19

(52) U.S. Cl. 424/63; 424/70.7

(57) ABSTRACT

The invention relates to compositions containing glitter containing dark colorant as well as to methods of applying such compositions to keratin materials and kits containing such compositions.

### FIELD OF THE INVENTION

The present invention generally relates to compositions comprising glitter comprising dark colorant as well as to methods of applying such compositions to keratin materials and kits comprising such compositions. Such compositions possess improved properties and characteristics such as, for example, enhancing the characteristics or properties of other glitter components in the same composition or in a complementary composition, for example by not interfering with or muting such characteristics or properties.

### BACKGROUND OF THE INVENTION

Cosmetic compositions often contain glitter. In the past, combining glitter into a cosmetic composition has been problematic, with the glitter properties of these materials diminishing, weakening or disappearing upon application. Thus, there is a need for improved glitter-containing compositions in which the glitter components maintain or improve their glitter properties or characteristics upon application by the end user.

Accordingly, one aspect of the present invention is a care and/or makeup and/or treatment composition for keratin materials such as eyelids or eyelashes which is able to address or overcome the aforementioned problems with the prior art compositions.

### BRIEF SUMMARY OF THE INVENTION

The present invention relates to compositions comprising glitter comprising dark colorant. Such a composition could be, for example, a topcoat which could be applied to a basecoat (for example, mascara).

The present invention further relates to compositions comprising glitter comprising dark colorant and at least one non-dark glitter.

The present invention also relates to compositions for eyelashes such as mascaras, topcoats and basecoats comprising glitter comprising dark colorant and, optionally, further comprising at least one non-dark glitter.

The present invention further relates to methods of applying at least two glitters to a keratin material comprising applying to the keratin material a glitter comprising dark colorant and at least one non-dark glitter.

The present invention also relates to methods of applying glitter to a keratin material comprising applying to the keratin material a basecoat composition comprising at least one non-dark glitter, and then applying to the basecoat composition a topcoat composition comprising glitter comprising dark colorant.

The present invention also relates to methods of applying glitter to a keratin material comprising applying to the keratin material a basecoat composition comprising glitter comprising dark colorant, and then applying to the basecoat composition a topcoat composition comprising at least one non-dark glitter.

The present invention also relates to methods of enhancing the glitter properties of non-dark glitter comprising applying to a keratin material a basecoat composition comprising at least one non-dark glitter, and then applying to the basecoat composition a topcoat composition comprising glitter comprising dark colorant.

The present invention also relates to methods of enhancing the glitter properties of non-dark glitter comprising applying to a keratin material a basecoat composition comprising glitter comprising dark colorant, and then applying to the basecoat composition a topcoat composition comprising at least one non-dark glitter.

The present invention further relates to methods of enhancing the glitter properties of non-dark glitter comprising combining the glitter with glitter comprising dark colorant.

The present invention also relates to methods of treating, caring for, making up or enhancing the appearance of keratin materials comprising applying compositions of the present invention to the keratin materials in an amount sufficient to treat, care for, make-up and/or enhance the appearance of the keratin materials.

The present invention also relates to kits comprising (a) a basecoat composition comprising at least one non-dark glitter; and (b) a topcoat composition comprising glitter comprising dark colorant.

The present invention also relates to kits comprising (a) a basecoat composition comprising glitter comprising dark colorant; and (b) a topcoat composition comprising at least one non-dark glitter.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Cosmetically acceptable” means that the item in question is compatible with any keratin material. For example, “cosmetically acceptable medium” means a medium that is compatible with any keratin material.

“Keratin material” includes, for example, skin, hair, nails, eyelashes, eyelids, eyebrows, lips and any other area of body or facial skin.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human lips followed by “kissing” a material, for example, a sheet of paper, after expiration of a certain amount of time following application, such as 2 minutes after application. Similarly, transfer resistance of a composition may be evaluated by the amount of product transferred from a wearer to any other substrate, such as transfer from the neck of an individual to a collar after the expiration of a certain amount of time following application. The amount of composition transferred to the substrate (e.g., collar, or paper) may then be evaluated and compared. For example, a composition may be transfer resistant if a majority of the product is left on the wearer, e.g., lips, neck, etc. Further, the amount transferred may be compared with that transferred by other compositions, such as commercially available compositions.

“Long wear” compositions as used herein, refers to compositions where at least one property chosen from consistency, texture, and color remains the same as at the time of application, as viewed by the naked eye, after an extended period of time, such as, for example, 1 hour, 2 hours, and further such as 8 hours. Long wear properties may be evaluated by any method known in the art for evaluating such properties. For example, long wear may be evaluated by a test involving the application of a composition to human skin (including lips) and evaluating the consistency, texture and color of the composition after an extended period of time. For example, the consistency, texture and color of a lip composition may be evaluated immediately following application and these characteristics may then be re-evaluated and compared after an individual has worn the lip composition for a certain amount of time. Further, these characteristics may be evaluated with respect to other compositions, such as commercially available compositions.

“Waterproof” as used herein refers to the ability to repel water and permanence with respect to water. Waterproof properties may be evaluated by any method known in the art for evaluating such properties. For example, a mascara composition may be applied to false eyelashes, which may then be placed in water for a certain amount of time, such as, for example, 20 minutes. Upon expiration of the pre-ascertained amount of time, the false eyelashes may be removed from the water and passed over a material, such as, for example, a sheet of paper. The extent of residue left on the material may then be evaluated and compared with other compositions, such as, for example, commercially available compositions. Similarly, for example, a composition may be applied to skin, and the skin may be submerged in water for a certain amount of time. The amount of composition remaining on the skin after the pre-ascertained amount of time may then be evaluated and compared. For example, a composition may be waterproof if a majority of the product is left on the wearer, e.g., eyelashes, skin, etc.

The compositions, methods and kits of the present invention can comprise, consist of, or consist essentially of the essential elements and limitations of the invention described herein, as well as any additional or optional ingredients, components, or limitations described herein or any otherwise useful ingredient found in personal care compositions intended for application to keratin materials.

The composition of the present invention may be in any form suitable for use on keratin materials such as, for example, non-solid anhydrous, oil-free or emulsion compositions (for example, water-in-oil emulsion, oil-in-water emulsion, multiple emulsion (W/O/W or O/W/O), suspensions, dispersions, nanoemulsions, etc.). The compositions of the present invention can be mascaras or eyeliners. Generally speaking, mascaras and eyeliners contain colorants such as pigments. The compositions of the present invention can be used as a basecoat and/or topcoat for application beneath and/or onto other products applied to keratin materials such as eyelashes or eyelids.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion. The stability is further tested by repeating the 8-week test at 4° C., 37° C., 45° C. and/or under freeze-thaw conditions. A composition is considered to lack stability if in any of these tests an abnormality that impedes functioning of the composition is observed. The skilled artisan will readily recognize an abnormality that impedes functioning of a composition based on the intended application.

“Glitter” is material which reflects flashes of light and, thus, appears to sparkle. Typically, glitter comprises a plurality of particles (i.e., pieces or fragments of a material) having a regular or irregular periphery, which reflects or refracts light. Preferably, glitter is coated with a transparent or translucent coating. Materials useful as glitter include particles of metal (e.g., aluminum, copper, silver, gold, and brass), particles of transparent or colored, solid organic materials (e.g., polyethylene terephthalate, polymethacrylate, and polyvinylbutyral), particles of metal coated film or paper (e.g., aluminum coated polyethylene terephthalate film) and metal film or paper coated with metal (e.g., polyethylene terephthalate film coated with aluminum). Glitter may be clear and/or be provided in a variety of colors (e.g., silver, gold, blue, red, etc.), or mixtures thereof; and may be provided in a variety of shapes (e.g., circles, squares, rectangles, triangles, diamonds, hexagons, stars, symbols, alphanumerics (i.e., letters and/or numbers), or mixtures of different shapes and sizes.

Compositions Comprising Glitter Comprising Dark Colorant

According to the present invention, compositions comprising glitter comprising dark colorant are provided. In accordance with the present invention, the glitter can contain any suitable glitter material or any suitable mixture of glitter materials. For example, the glitter may comprise a metal, an organic material, or mixtures thereof. Particularly preferred metals include aluminum and silver.

Also in accordance with the present invention, the glitter comprises a dark colorant. Any suitable, cosmetically acceptable dark colorant can be used. “Dark colorant” means any colorant having an L* value of 30 or less according to the CIELAB scale based on the Opponent-Colors Theory and discussed in Application Notes (Jul. 1-15, 1996 and Aug. 1-15, 1996) and in CIE Publication No. 15, Colorimetry, Supplement No. 2, the entire contents of which are hereby incorporated by reference in their entirety. According to the CIELAB scale which ranges from 0 to 100, “L*” indicates the level of light or dark in a color.

Thus, in accordance with the present invention, the glitter can comprise a colorant of any color such as, for example, black, gray, brown, purple, green, etc. as long as the L* value associated with the colorant is 30 or less, preferably 25 or less, more preferably 20 or less, and most preferably 15 or less.

According to preferred embodiments, the glitter comprises a black colorant. A particularly preferred black colorant is D&C Black No. 2 (CI 77266).

Further in accordance with preferred embodiments, the glitter is coated. Any suitable coating material(s) can be used as long as the material is cosmetically acceptable, forms an acceptable coating on or around the glitter, and permits sufficient light to pass through it so that the glitter material has glitter properties. Preferably, the coating material is transparent or substantially transparent and/or comprises a film forming agent (film former) such as, for example, polyethylene terephthalate or polymethacrylate. The coating can be in a single layer of one or more coating materials, or in multiple layers of two or more coating materials.

According to preferred embodiments, the coating material comprises a polyurethane compound. Polyurethane compounds are well-known compounds which, generally speaking, are produced by reacting a polyol with a diisocyanate or a polymeric isocyanate. In accordance with the present invention, any polyurethane compound which is capable of forming a coating on or around glitter material and which is cosmetically acceptable can be used. For example, suitable polyurethane compounds include, but are not limited to, polyurethane-1, polyurethane-2, polyurethane-3, polyurethane-4, polyurethane-5, polyurethane-6, polyurethane-7, polyurethane-8, polyurethane-9, polyurethane-10, polyurethane-11, polyurethane-12, polyurethane-13, polyurethane-14, polyurethane-15, polyurethane-16, polyurethane-17, polyurethane-18, polyurethane-19 and polyurethane-20. Polyurethane-11 is particularly preferred.

According to preferred embodiments, the glitter coating comprises at least some of the black colorant. Most preferably, the glitter coating contains essentially all of the black colorant (that is, 95% or more of the black colorant) or all of the black colorant.

A particularly preferred example of glitter comprising dark colorant is CosmetaGem which is commercially available from Glitterex. CosmetaGem comprises aluminum surrounded by a polyethylene terephthalate film which, in turn, is surrounded by polyurethane-11. D&C Black No. 2 is dispersed in the coating around the aluminum. Another suitable coated glitter is Brilliant Jet Black (Polyester Jewels) from Meadowbrook Inventions, Inc., in Bernardsville, N.J.

According to one embodiment of the present invention, compositions comprising glitter comprising dark colorant further comprise at least one non-dark glitter. Thus, according to this embodiment, the compositions comprise at least two different glitters. In accordance with this embodiment, the “non-dark glitter” can be any glitter (as discussed above) having a CIELAB scale L* value of more than 30, preferably more than 40, and most preferably from 50-100. Suitable examples include, but are not limited to, those glitters produced by Glitterex or Meadowbrook (Alpha Jewels, Crystalina, PolyFlake, Hologram, etc.).

According to this embodiment, the composition comprises glitter comprising dark colorant in an amount sufficient to enhance (that is, to improve or, at least, to not impede) the glitter properties of the non-dark glitter. Preferably, the weight ratio of glitter comprising dark colorant to non-dark glitter is from about 1:3 to about 3:1, more preferably from about 1:2 to about 2:1.

According to another embodiment of the present invention, compositions comprising glitter comprising dark colorant are free or substantially free of non-dark glitter.

According to this embodiment, the composition contains either no non-dark glitter (that is, the composition is free of non-dark glitter) or it contains so little non-dark glitter that it does not impart any non-dark color characteristics to the composition (that is, the composition is substantially free of non-dark glitter). Preferably, such compositions are basecoat compositions which are applied to a keratin material before application of a topcoat composition (as discussed below), although such compositions could also be topcoat compositions, if desired.

In accordance with the present invention, the total amount of glitter (including both glitter comprising dark colorant and non-dark glitter, if present) preferably ranges from about 0.1% to about 100% by weight with respect to the total weight of the composition. The total amount of glitter present will, of course, depend upon composition type. For example, loose powder compositions could contain up to about 100% glitter, compact powder compositions could contain up to about 80% or 90% glitter, fingernail polish could contain up to about 50% glitter, mascara could contain up to about 30% glitter, and so on.

Thus, generally speaking, the total amount of glitter present in the composition preferably ranges from about 0.5% to about 85% by weight with respect to the total weight of the composition, more preferably from about 0.75% to about 80% by weight with respect to the total weight of the composition, more preferably from about 1% to about 50% by weight with respect to the total weight of the composition, and more preferably from about 10% to about 30% by weight with respect to the total weight of the composition, including all ranges and subranges therebetween.

Additional Ingredients

The compositions of the present invention can also comprise any additive usually used in cosmetic or dermatologic compositions. For example, waxes, film forming agents, dispersants, antioxidants, oils, preserving agents, fragrances, fillers, neutralizing agents, surfactants, cosmetic and dermatological active agents such as, for example, emollients, moisturizers, vitamins, anti-wrinkle agents, essential fatty acids, and sunscreens, and mixtures thereof can be added. A non-exhaustive listing of such ingredients can be found in U.S. patent application Ser. No. 10/733,467, filed Dec. 12, 2003, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelashes of human beings.

Specific examples of additional ingredients include oils, particularly if the composition is an anhydrous composition or an emulsion. Any oils can be used in accordance with the present invention. The oils can be volatile or non-volatile, silicone-based and/or hydrocarbon-based, etc. Thus, for example, the external oil phase may contain, independently or in combination, volatile silicone oils, non-volatile silicone oils, volatile non-silicone oils and non-volatile non-silicone oils.

In one embodiment, the compositions of the present invention are substantially free of silicone oils (i.e., contain less than about 1% of silicone oil). In another embodiment, the compositions are substantially free of non-silicone oils (i.e., contain less than about 1% of non-silicone oil). In another embodiment, the compositions are substantially free of non-volatile oils (i.e., contain less than about 1% of non-volatile oil). In yet another embodiment, the compositions are substantially free of volatile oils (i.e., contain less than about 1% of volatile oil).

Water, when present, preferably represents from about 1% to about 70% by weight of the total weight of the composition, more preferably from about 5% to about 60% of the total weight of the composition, and most preferably from about 10% to about 50%, including all ranges and subranges therebetween.

The compositions may also optionally comprise at least one non-glitter coloring agent. Suitable non-glitter coloring agents include but are not limited to pigments, dyes, such as liposoluble dyes, and nacreous pigments.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, may be present in the composition in a concentration ranging up to 50% by weight of the total weight of the composition, such as from 0.1% to 20%, preferably from 0.1% to 15%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0.5% to 40%, and further such as from 2% to 30%, including all ranges and subranges therebetween. In the case of certain products, the pigments, including nacreous pigments, may, for example, represent up to 50% by weight of the composition.

According to preferred embodiments of the present invention, methods of applying at least two glitters to a keratin material (such as, for example, skin, hair, nails, eyelashes, eyelids, eyebrows, lips) comprising applying to the keratin material a glitter comprising dark colorant and at least one non-dark glitter are provided. According to particularly preferred embodiments, the glitter comprising dark colorant and at least one non-dark glitter are in a single composition (as described above) and, thus, their application occurs at the same time when the composition is applied to the keratin material.

According to other preferred embodiments, the glitter comprising dark colorant and at least one non-dark glitter are in separate compositions and the compositions are applied to the keratin material sequentially (that is, one after the other). For example, the composition comprising the glitter comprising dark colorant can be applied to the keratin material before the composition comprising the at least one non-dark glitter, or vice versa.

According to such preferred embodiments, methods of applying glitter to a keratin material (such as, for example, skin, hair, nails, eyelashes, eyelids, eyebrows, lips) comprising applying to the keratin material a basecoat composition comprising glitter comprising dark colorant, and then applying to the basecoat composition a topcoat composition comprising at least one non-dark glitter are provided.

According to yet other preferred embodiments of the present invention, methods of enhancing (that is, improving or, at least, not impeding) the glitter properties of at least one non-dark glitter comprising combining such glitter with glitter comprising dark colorant are provided.

According to one aspect of this embodiment, the at least one non-dark glitter and the glitter comprising dark colorant are combined in a single composition.

According to another aspect of this embodiment, the at least one non-dark glitter and the glitter comprising dark colorant are combined by applying to a keratin material (such as, for example, skin, hair, nails, eyelashes, eyelids, eyebrows, lips) a basecoat composition comprising glitter comprising a dark colorant, and then applying to the basecoat composition a topcoat composition comprising at least one non-dark glitter.

According to still other preferred embodiments of the present invention, methods of treating, caring for, making up or enhancing the appearance of keratin materials (such as, for example, skin, hair, nails, eyelashes, eyelids, eyebrows, lips) comprising applying compositions of the present invention to the keratin materials in an amount sufficient to treat, care for, make-up and/or enhance the appearance of the keratin materials are provided.

In accordance with all of the foregoing embodiments of the present invention, the compositions of the present invention may be applied to keratin materials as needed, preferably once or twice daily, more preferably once daily and then preferably allowed to dry before subjecting to contact such as with clothing or other objects.

The present invention also envisages kits and/or prepackaged materials suitable for consumer use containing one or more compositions according to the description herein (for example, kits containing (1) a composition for application to keratin material such as mascara, eyeliner, a lip composition such as a lipstick or liquid lip color, foundation, etc.; and (2) a basecoat and/or topcoat). According to particularly preferred embodiments, kits comprising (a) a basecoat composition comprising glitter comprising a dark colorant; and (b) a topcoat composition comprising at least one non-dark glitter are provided. Such kits may also include other compositions or components such as, for example, a cosmetic removing composition, instructions for applying or using the compositions in the kit, cosmetic application devices (for example, a mascara brush), etc.

The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### EXAMPLE 1

### Eyelash Composition

| | INCI NAME | |
|---|---|---|
| A | Water | 60.01 |
| | Carbomer | 0.60 |
| | Disodium EDTA | 0.04 |
| B | Polyvinyl Alcohol | 0.50 |
| | Water | 10.00 |
| C | Panthenol | 0.10 |
| D | Polyethylene Terephthalate (and) Aluminum Powder | 9.00 |
| | [Glitter] | |
| | Polyethylene Terephthalate (and) Polyurethane-11 (and) | 4.00 |
| | Black 2 (and) Aluminum Powder [Glitter] | |
| E | Triethanolamine | 0.95 |
| | Water | 5.00 |
| F | Propylene Glycol | 2.10 |
| | Glycerin | 2.00 |
| G | Alcohol Denatured | 5.00 |
| | Methylparaben | 0.20 |
| | Phenoxyethanol | 0.50 |
| | TOTAL | 100.00 |

### EXAMPLE 2

### Eyelash Composition

| | INCI NAME | |
|---|---|---|
| A | Water | 57.01 |
| | Carbomer | 0.60 |
| | Disodium EDTA | 0.04 |
| B | Polyvinyl Alcohol | 0.50 |
| | Water | 10.00 |
| C | Panthenol | 0.10 |
| D | Polyethylene Terephthalate (and) Polyurethane-11 | 10.00 |
| | (and) Aluminum Powder [glitter] | |
| | Polyethylene Terephthalate (and) Polyurethane-11 | 6.00 |
| | (and) Black 2 (and) Aluminum Powder [Glitter] | |
| E | Triethanolamine | 0.95 |
| | Water | 5.00 |
| F | Propylene Glycol | 2.10 |
| | Glycerin | 2.00 |
| G | Alcohol Denatured | 5.00 |
| | Methylparaben | 0.20 |
| | Phenoxyethanol | 0.50 |
| | TOTAL | 100.00 |

### EXAMPLE 3

### Eyelash Composition

| | INCI NAME | |
|---|---|---|
| A | Water | 57.01 |
| | Carbomer | 0.60 |
| | Disodium EDTA | 0.04 |
| B | Polyvinyl Alcohol | 0.50 |
| | Water | 10.00 |
| C | Panthenol | 0.10 |
| D | Polyethylene Terephthalate (and) Polyurethane-11 (and) | 12.00 |
| | Yellow-5 (and) Aluminum Powder [Glitter] | |
| | Polyethylene Terephthalate (and) Polyurethane-11 (and) | 4.00 |
| | Black 2 (and) Aluminum Powder [Glitter] | |
| E | T.E.A | 0.95 |
| | Water | 5.00 |
| F | Propylene Glycol | 2.10 |
| | Glycerin | 2.00 |
| G | Alcohol Denatured | 5.00 |
| | Methylparaben | 0.20 |
| | Phenoxyethanol | 0.50 |
| | TOTAL | 100.00 |

What is claimed is:

1. A method of applying at least two glitters to a keratin material comprising applying to the keratin material a glitter comprising dark colorant and at least one non-dark glitter.

2. The method of claim 1, wherein the glitter comprising dark colorant comprises a coating comprising a polyurethane compound.

3. The method of claim 2, wherein the coating of the coated glitter comprises polyurethane-11.

4. The method of claim 1, wherein the glitter comprising dark colorant comprises a coating comprising polyethylene terephthalate.

5. The method of claim 3, wherein the coating of the coated glitter further comprises polyethylene terephthalate.

6. The method of claim 5, wherein the coating of the coated glitter further comprises black colorant.

7. The method of claim 1, wherein the glitter comprising dark colorant comprises at least one metal.

8. The method of claim 7, wherein the at least one metal is silver or aluminum.

9. The method of claim 6, wherein the glitter comprising dark colorant comprises at least one metal.

10. The method of claim 9, wherein the at least one metal is silver or aluminum.

11. The method of claim 1, wherein the keratin material is an eyelash or an eyelid.

12. The method of claim 1, wherein the glitter comprising dark colorant and the at least one non-dark glitter are in separate compositions and the compositions are applied to the keratin material sequentially.

13. The method of claim 12, wherein the composition comprising the glitter comprising dark colorant is applied to the keratin material before the composition comprising the at least one non-dark glitter.

14. The method of claim 12, wherein the composition comprising the at least one non-dark glitter further comprises at least one pigment.

15. The method of claim 12, wherein the composition comprising the glitter comprising dark colorant further comprises at least one pigment.

16. The method of claim 12, wherein the keratin material is an eyelash or an eyelid.

17. The method of claim 13, wherein the keratin material is an eyelash or an eyelid.

18. The method of claim 14, wherein the keratin material is an eyelash or an eyelid.

19. The method of claim 1, wherein the weight ratio of glitter comprising dark colorant to the at least one non-dark glitter applied to the keratin material is about 1:2 to about 2:1.

20. The method of claim 12, wherein the weight ratio of glitter comprising dark colorant to the at least one non-dark glitter applied to the keratin material is about 1:2 to about 2:1.

21. The method of claim 12, wherein the amount of glitter comprising dark colorant present in the composition comprising said glitter ranges from about 0.75% to about 80% by weight with respect to the total weight of the composition.

22. The method of claim 13, wherein the amount of glitter comprising dark colorant present in the composition comprising said glitter ranges from about 0.75% to about 80% by weight with respect to the total weight of the composition.

23. A basecoat composition comprising glitter comprising dark colorant.

24. The basecoat composition according to claim 23, wherein the dark colorant is black colorant.

25. A composition comprising a glitter comprising dark colorant and at least one non-dark glitter.

26. The composition according to claim 25, wherein the dark colorant is black colorant.

27. A kit comprising (a) a basecoat composition comprising glitter comprising dark colorant; and (b) a topcoat composition comprising at least one non-dark glitter.

28. A method of enhancing the glitter properties of a non-dark glitter comprising combining the non-dark glitter with a glitter comprising dark colorant.
